(19) United States
(12) Patent Application Publication
(10) Pub. No.: US 2018/0164995 A1
(43) Pub. Date: Jun. 14, 2018

(54) SYSTEM AND METHOD FOR PRESENTING A USER INTERFACE

(71) Applicant: General Electric Company, Schenectady, NY (US)

(72) Inventor: Balint Czupi, Seewalchen am Attersee (AT)

(21) Appl. No.: 15/377,896

(22) Filed: Dec. 13, 2016

(51) Int. Cl. G06F 3/0484; G06F 3/0481; G06F 3/0482; G06F 3/0488; A61B 8/00

(52) U.S. Cl. CPC G06F 3/04847; G06F 3/04817; A61B 8/00; G06F 3/04883; G06F 3/0482

(57) ABSTRACT

Methods and systems are provided for presenting a user interface element for both unlocking an imaging system and initiating a selected operation of the imaging system via a single user input. In one embodiment, an imaging system comprises a touch-sensitive display device, a controller, and a storage device storing instructions executable by the controller to display, via the touch-sensitive display device, a user interface comprising a central user interface element and a plurality of operation indicators positioned around a periphery of the central user interface element and, responsive to user input moving the central user interface element in a direction toward a first indicator of the operation indicators, executing an application or operation associated with the first indicator upon intersecting a selection region for the first indicator.

### FIELD

Embodiments of the subject matter disclosed herein relate to medical imaging and techniques for displaying a user interface for interacting with medical imaging devices and systems.

### BACKGROUND

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device. In one example, the display device may be a touch-sensitive display, also referred to as a touchscreen. A user may interact with the touchscreen to analyze the displayed image. For example, a user may use their fingers on the touchscreen to position a region of interest (ROI), place measurement calipers, or the like. The workstation or device may also include hardware actuators, such as buttons, knobs, dials, etc. A user may interact with the hardware actuators to initiate operations of the ultrasound imaging system. For example, a user may interact with hardware actuators to initiate a scan, set up a scan (e.g., select a scanning mode), and/or execute other operations or applications using the ultrasound imaging system.

In order to avoid accidental selection of operating inputs when the ultrasound system is not in use, the system may utilize a sleep or lock state that is triggered responsive to a period of inactivity that exceeds a threshold or receipt of a specific input to enter the sleep or lock state. The sleep or lock state may also be triggered responsive to a startup of the ultrasound imaging system. During the sleep or lock state, a generic lock screen and/or a blank screen may be displayed until a user provides an input to wake up or unlock the system. For example, the input may be an authorization input or other request to wake up or unlock the system. In some examples, any input to the system may serve to first wake up or unlock the system, and further inputs may be used to operate the system.

However, the inventors have recognized challenges with such touch- and hardware actuator-based user interfaces for ultrasound imaging systems. For example, when the system is locked or in a sleep mode, a user may need to provide one or more inputs to wake/unlock the system before the user is able to provide an instruction for the system to perform an action. Such a delay may be exacerbated if the inputs usable to wake up or unlock the system are not made clear to the user. For example, if a blank or generic screen is presented by the system during the sleep or locked state, the user may not be sure of which inputs will wake/unlock the system, and may hesitate and/or provide incorrect inputs (e.g., inputs that do not wake/unlock the system), leading to user frustration and delays in system operation.

### BRIEF DESCRIPTION

In one embodiment, an imaging system comprises a touch-sensitive display device, a controller, and a storage device storing instructions executable by the controller to display, via the touch-sensitive display device, a user interface comprising a central user interface element and a plurality of operation indicators positioned around a periphery of the central user interface element and, responsive to user input moving the central user interface element in a direction toward a first indicator of the operation indicators, executing an application or operation associated with the first indicator upon intersecting a selection region for the first indicator.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasonic imaging system according to an embodiment of the invention.

FIGS. 2A-6 show example unlock elements for a user interface of an imaging system according to embodiments of the invention.

FIGS. 7A and 7B show example screensaver images for a display of an imaging system according to embodiments of the invention.

FIGS. 8-10 show flow charts for example methods of operating an imaging system and associated touchscreen during different operating conditions according to embodiments of the invention.

FIG. 11 schematically shows example input and output mechanisms for an imaging system according to an embodiment of the invention.

### DETAILED DESCRIPTION

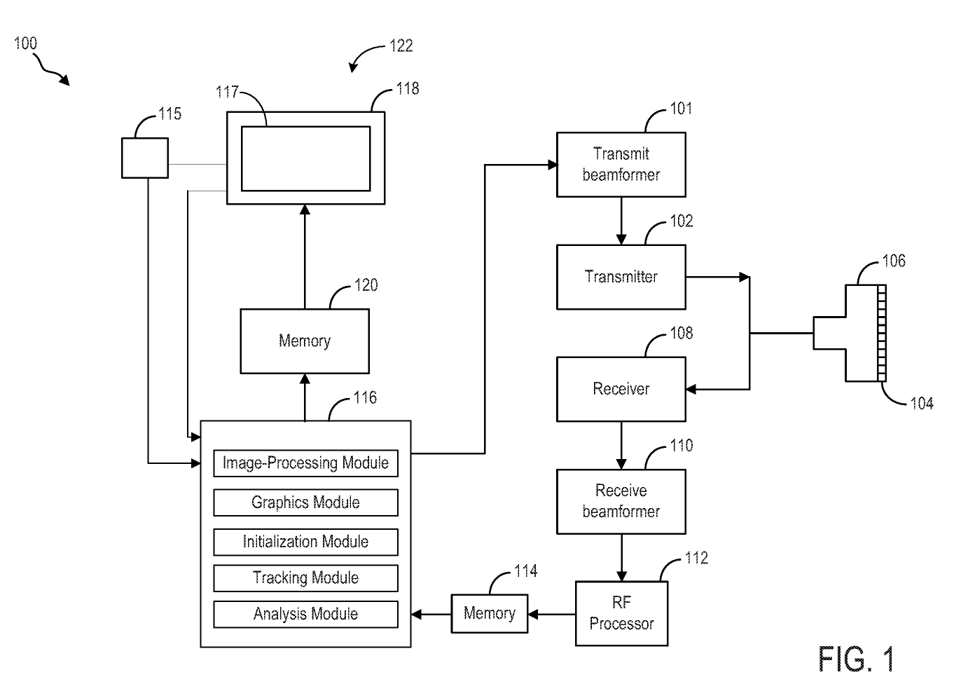

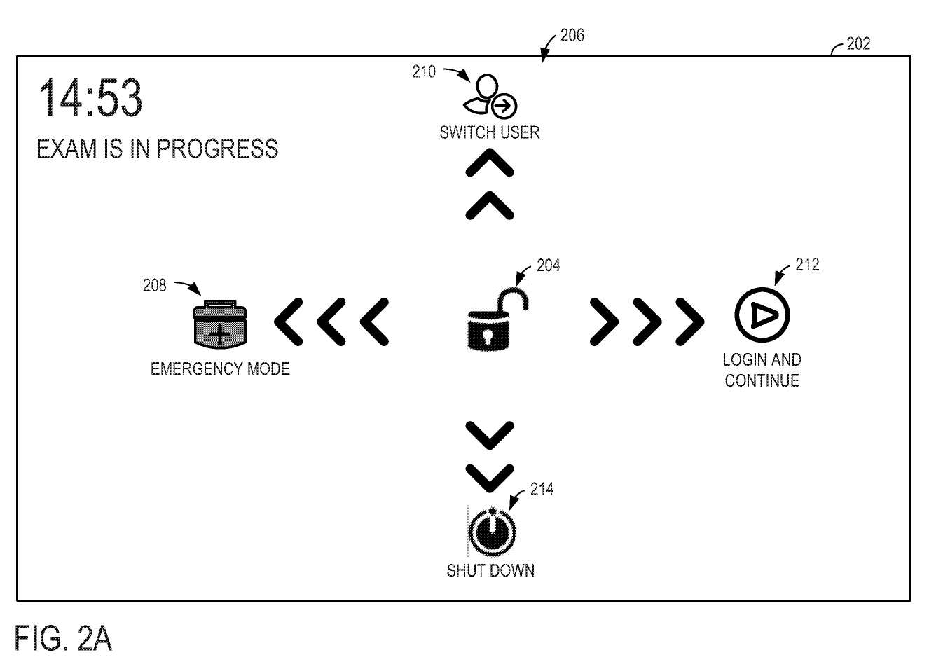

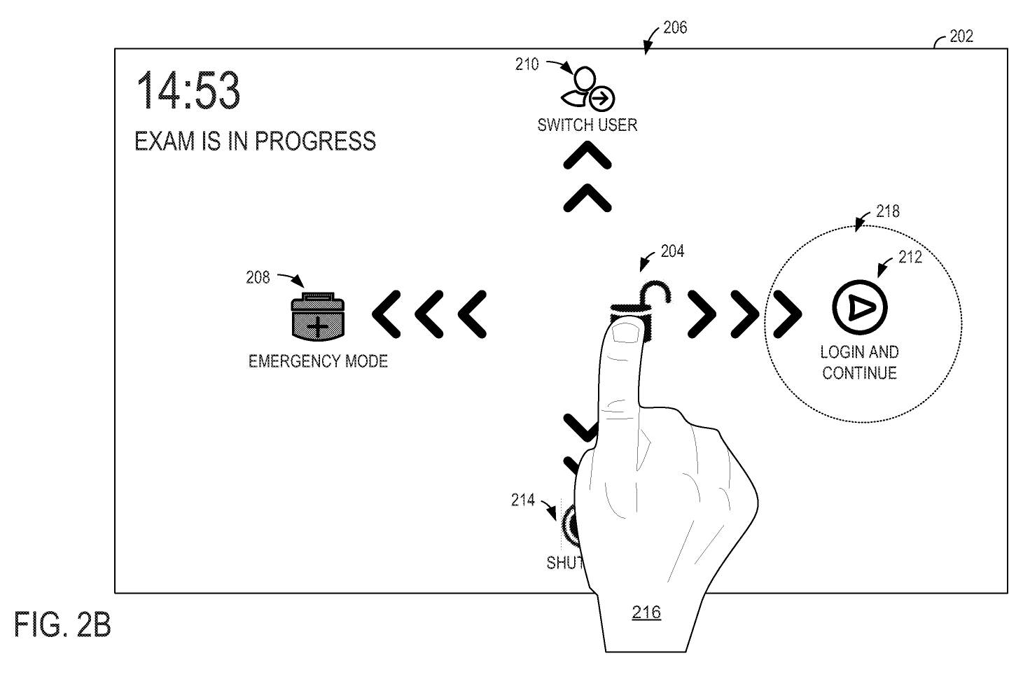

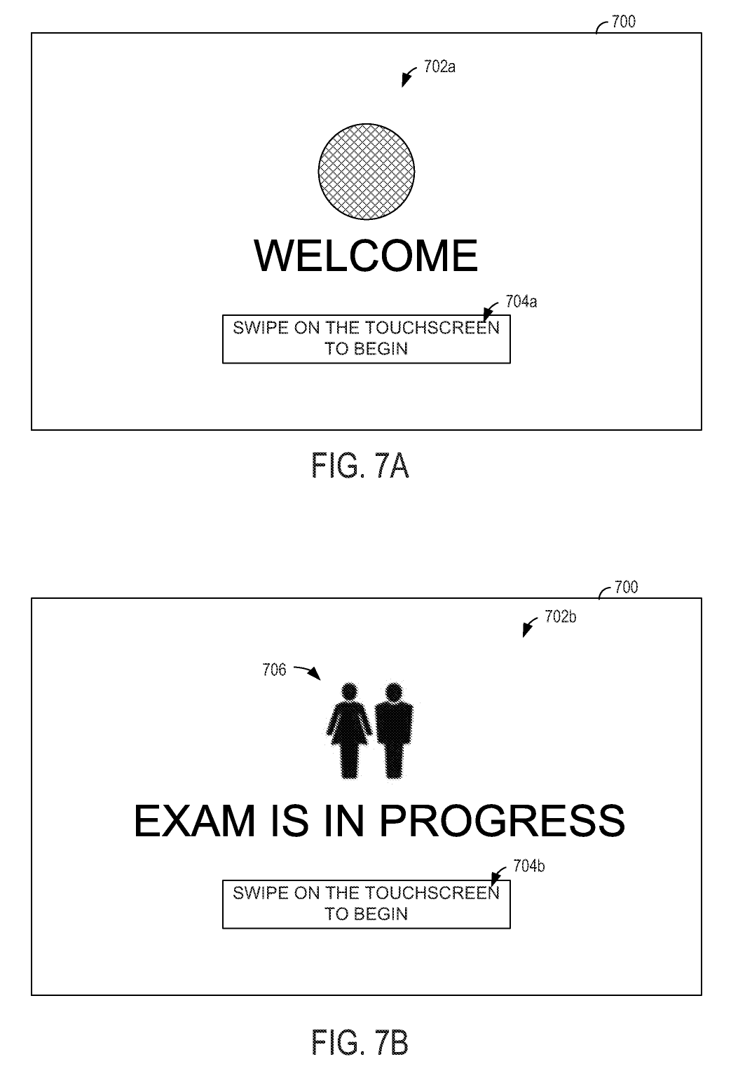

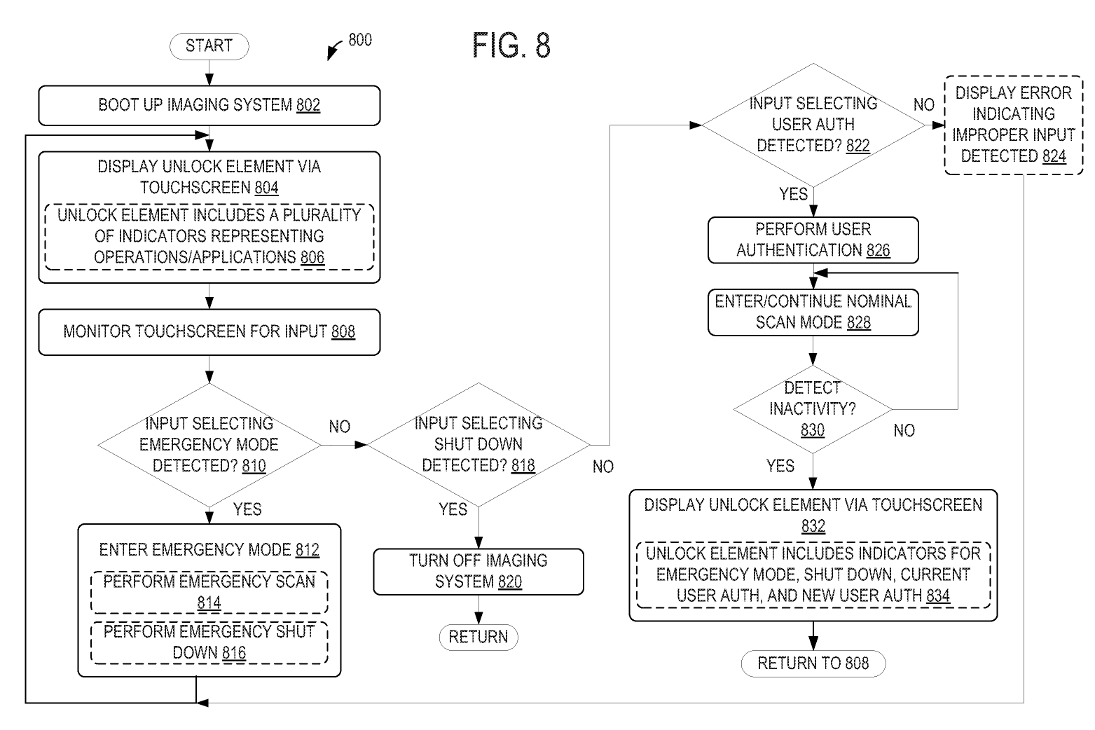

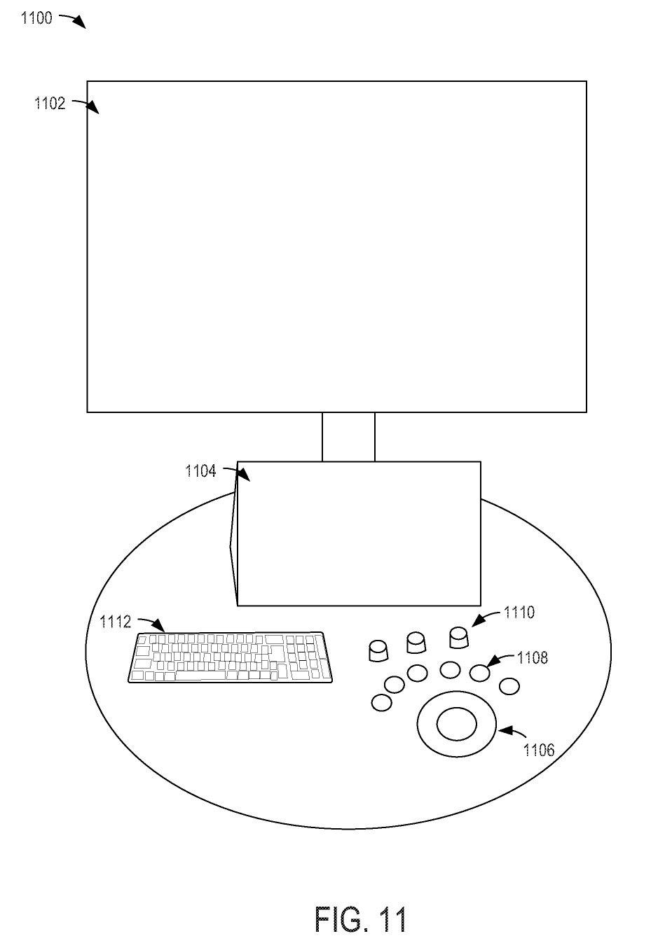

The following description relates to various embodiments of an imaging system, such as the ultrasound imaging system shown in FIG. 1. In particular, systems and methods are described for providing a lock screen user interface that enables different unlock mechanisms for different inputs (e.g., for different gesture and/or touch inputs). Though the systems and methods described below for outputting a gesture-responsive lock screen interface via a touch-sensitive display are discussed with reference to an ultrasound imaging system, it should be noted that the methods described herein may be applied to a plurality of imaging systems (e.g., Mill, PET, X-ray, etc.). As shown in FIGS. 2A-6, different unlock elements of a user interface may be provided to enable a user to unlock the imaging system and directly access a selected application or operate according to a selected mode. FIGS. 7A and 7B show example screensaver images for another display of the imaging system, which may be presented while the unlock element is being displayed via the other displays. As shown in FIGS. 8-10, different unlock elements may be presented responsive to different conditions of the imaging system and/or user of the imaging system. As shown in FIG. 11, an imaging system may include various input mechanisms, which may be used to operate the imaging system under different conditions.

FIG. 1 illustrates a block diagram of a system 100 according to one embodiment. In the illustrated embodiment, the system 100 is an imaging system and, more specifically, an ultrasound imaging system. However, it is understood that embodiments set forth herein may be implemented using other types of medical imaging modalities (e.g., MR, CT, PET/CT, SPECT, etc.). Furthermore, it is understood that other embodiments do not actively acquire medical images. Instead, embodiments may retrieve image data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart.

In the illustrated embodiment, the system 100 includes a transmit beamformer 101 and transmitter 102 that drives an array of elements 104, for example, piezoelectric crystals, within a diagnostic ultrasound probe 106 (or transducer) to emit pulsed ultrasonic signals into a body or volume (not shown) of a subject. The elements 104 and the probe 106 may have a variety of geometries. The ultrasonic signals are back-scattered from structures in the body, for example, blood vessels and surrounding tissue, to produce echoes that return to the elements 104. The echoes are received by a receiver 108. The received echoes are provided to a receive beamformer 110 that performs beamforming and outputs an RF signal. The RF signal is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage). The system 100 also includes a system controller 116 that includes a plurality of modules, which may be part of a single processing unit (e.g., processor) or distributed across multiple processing units. The system controller 116 is configured to control operation of the system 100. For example, the system controller 116 may include an image-processing module that receives image data (e.g., ultrasound signals in the form of RF signal data or IQ data pairs) and processes image data. For example, the image-processing module may process the ultrasound signals to generate slices or frames of ultrasound information (e.g., ultrasound images) for displaying to the operator. When the system 100 is an ultrasound system, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D) or three-dimensional (3D). When multiple two-dimensional (2D) images are obtained, the image-processing module may also be configured to stabilize or register the images.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices of acquired ultrasound information that are not scheduled to be displayed immediately. The image memory 120 may comprise any known data storage medium, for example, a permanent storage medium, removable storage medium, and the like. Additionally, the image memory 120 may be a non-transitory storage medium.

In operation, an ultrasound system may acquire data, for example, volumetric data sets by various techniques (for example, 3D scanning, real-time 3D imaging, volume scanning, 2D scanning with probes having positioning sensors, freehand scanning using a voxel correlation technique, scanning using 2D or matrix array probes, and the like). Ultrasound images of the system 100 may be generated from the acquired data (at the controller 116) and displayed to the operator or user on the display device 118.

The system controller 116 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information from the controller 116 to the operator by displaying the information to the operator. The display device 118 and/or the user interface 122 may also communicative audibly. The display device 118 is configured to present information to the operator during the imaging session. The information presented may include ultrasound images, graphical elements, user-selectable elements, and other information (e.g., administrative information, personal information of the patient, and the like).

In some embodiments, the user interface 122 may also include one or more user interface input devices 115, such as a physical keyboard, mouse, and/or touchpad. In one embodiment, a touchpad may be configured to the system controller 116 and display area 117, such that when a user moves a finger/glove/stylus across the face of the touchpad, a cursor atop the ultrasound image on the display area 117 moves in a corresponding manner. In additional or alternative embodiments, input device 115 may include a touch-sensitive display (e.g., a touchscreen). In such embodiments, a display area of input device 115 may be controlled to present the same or different information than the display area 117 of display device 118. For example, in a locked or sleep state of the imaging system 100, a display area of input device 115 may present a user interface for use in unlocking and/or waking the imaging system (as will be described in more detail below), while the display area 117 of the display device 118 may present a generic screensaver, a blank screen, and/or other different display (e.g., a non-interactive display). During an examination or other scanning operation of the imaging system (e.g., when the device is unlocked/awake), one or both of the display areas of display device 118 and input device 115 may be interactive to enable a user to provide input during the examination/scanning operation. In examples where different user interface elements are provided for the display device 118 and the input device 115, the input device 115 may be controlled to provide a different type of user interface than the display device 118. For example, the display device 118 may provide a user interface for annotated or adjusting scanned images during an examination, while the input device 115 may provide a user interface for controlling the examination and/or state of the imaging system (e.g., selectable options for changing a mode of operation of the imaging system, for authenticating or logging in a different user to the imaging system, etc.).

In addition to the image-processing module, the system controller 116 may also include a graphics module, an initialization module, a tracking module, and an analysis module. The image-processing module, the graphics module, the initialization module, the tracking module, and the analysis module may coordinate with one another to present information to the operator during and/or after the imaging session. For example, the image-processing module may be configured to display an acquired image on the display device 118, and the graphics module may be configured to display designated graphics along with the ultrasound image, such as graphical outlines, which represent lumens or vessel walls in the acquired image. The image-processing and/or graphics modules within the system controller 116, may also be configured to generate a 3D rendering or image (not shown) of the entire vascular structure. In some embodiments the system controller 116 may also house an image-recognition module (not shown), which accesses stored images/videos (i.e., an image library) from either or both of the memory 114 and the memory 120, before analyzing them. For example, knowing the parameters under which a protocol is being carried out (ultrasound type, scan plane, tissue being imaged, etc.) the image recognition module may compare a live image on the display area 117, to one stored in memory 120, in order to analyze the image and thereby improve the accuracy of placing and utilizing analytical tools. In an alternative embodiment, instead of utilizing an image recognition module and image library, the system controller may house instructions for analyzing acquired imaging date (e.g., ultrasound images/videos acquired with the probe) and automatically determining a desired placement of one or more analytical tools. For example, the controller may include algorithms stored within a memory of the controller for analyzing an acquired image and determining placement of an analytical tool, such as an ROI (a region of interest). In yet another embodiment, the system controller may utilize both an image recognition module (also referred to herein as stored image data) and separate instructions for analyzing the displayed image/video apart from an image library, and both of these approaches may be used to increase the accuracy of placing and utilizing analytical tools.

During an examination or other scanning operation, the screen of the display area 117 of the display device 118 may be made up of a series of pixels which display the data acquired with the probe 106. The acquired data includes one or more imaging parameters calculated for each pixel, or group of pixels (for example, a group of pixels assigned the same parameter value), of the display, where the one or more calculated image parameters includes one or more of an intensity, velocity, color flow velocity, texture, graininess, contractility, deformation, and rate of deformation value. The series of pixels then make up the displayed image generated from the acquired ultrasound data. As mentioned above, the data acquired with the probe 106 and processed by the controller 116 may be 2D or 3D data. For example, traditionally, B-mode images, otherwise known as 2D images may be generated from A-mode information. A mode, where A stands for amplitude, is information of the reflected signal in a single ultrasound beam that is continually displayed as distance from the probe and intensity, are shown by position and amplitude in a line on an oscilloscope. A-mode information from many beams typically form a sector in a plane of the body, which is then shown as pixel intensity on a monitor, which is known as B-mode, where B stands for brightness. B mode may be used for anatomic assessment and orientation in the body, also for localizing and as a background display of other information such as Doppler signals. As such, B mode (2D) information may be used to identify a feature of interest and subsequently position a region of interest (ROI) that can then be manipulated for analysis of image content. As used herein, ROI refers to a border that either partially or fully encapsulates a feature of interest (such as a target tissue, organ, vessel lumen, tumor, etc.). In one embodiment, an ROI may be a user placed outline along the border of a feature of interest. In an alternate embodiment, an ROI may be defined as a moveable box overlaying an acquired ultrasound image where color flow data is acquired.

Returning to B mode imaging, some manipulations may include implementation of an ROI around the feature of interest which is then subjected to image data content analysis, contrast intensity analysis, color Doppler velocity analysis, grayscale, calculation of mean, median and standard deviation of intensity per frame, graphical display of time vs intensity data, etc.

Placement of measurement calipers in 2D medical imaging may be used for acquiring measurement values of a feature of interest (e.g., fetus, tumor, organ etc.). Measurements can then be used in determining what stage of gestation a fetus is currently in, if a tumor is growing/shrinking, if an organ is unusually large due to inflammation, along with a plethora of additional calculations dependent on measurement values to produce an accurate diagnosis. It should be noted that being able to correctly identify a feature of interest within an image requires successful interpretation of pixel variances, that is to say, the user must be able to clearly identify the borders between varying anatomical features, referred to as line delineation. Successful line delineation allows differentiation between anatomical features, correct placement of ROI borders, and ultimately aids in whatever diagnosis can be made from the 2D imaging at hand.

A 3D medical imaging dataset acquired with the probe 106 includes a volume dataset including a plurality of voxels. Each voxel, or volume-element, is assigned a value or intensity. Additionally, each voxel may be assigned an opacity as well. The value or intensity may be mapped to a color according to some embodiments. As one example, a volume-rendered image may be generated from the 3D dataset using a ray casting technique. For example, the controller 116 may cast a plurality of parallel rays from a view plane of the display 118 (which comprises the series of pixels) through the 3D medical imaging dataset. It should be appreciated that multiple rays may be cast in order to assign values to all of the pixels within the view plane. The controller 116 may use a “front-to-back” or a “back-to-front” technique for volume composition in order to assign a value to each pixel in the view plane that is intersected by the ray. For example, starting at the front, that is the direction from which the image is viewed, the intensities of all the voxels along the corresponding ray may be summed. An opacity value, which corresponds to light attenuation, is assigned to each voxel. The intensity is multiplied by the opacity of the voxels along the ray to generate an opacity-weighted value. These opacity-weighted values are then accumulated in a front-to-back or in a back-to-front direction along each of the rays. The process of accumulating values is repeated for each of the pixels in the view plane in order to generate a volume-rendered image. In this way, each pixel used to form the image displayed on the display 118 may have an intensity, or brightness value associated with it.

In some examples, the display device 118 and/or the input device 115 is adapted to be touch-sensitive and may provide tactile feedback to the user. The touch-sensitive display device 118 and/or input device 115 may be capable of communicating with the controller 116 in order to deliver tactile feedback representing various structures and features of the image on the display area 117 of the display device 118. The feedback provided via the display device 118 and/or input device 115 may additionally or alternatively indicate a level of completion of an input gesture, an error with an input gesture, and/or otherwise provide information regarding input provided by the user and detected by the respective touch-sensitive mechanism of the display device 118/input device 115. For example, input to unlock the imaging system may include performing a touch-based gesture including swiping a user's finger (or other input device) across a touch sensitive surface of the display device/input device. As will be described in more detail below, different operations may be performed based on a direction of the swiping, and tactile feedback may be provided based on a direction of the swiping and/or a progression of the swiping. For example, tactile feedback may be configured to increase in intensity (e.g., providing a greater amplitude and/or frequency of vibratory output) as an input gesture nears a selectable user interface element. In other examples, tactile feedback (or different tactile feedback) may be provided responsive to detecting a gesture input that does not follow a predefined input path. For example, an unlock user interface element may include end selectable elements positioned on two opposing sides of a middle selectable element, such that dragging the middle selectable element toward one of the two end selectable elements results in the selection of that end selectable element. In such an example, tactile feedback may be provided if movement of the middle selectable element is directed away from the two end selectable elements (e.g., if the two end selectable elements are located along a same axis, moving the middle selectable element in a direction approximately perpendicular to the axis may constitute moving the middle selectable element “away” from both of the end selectable elements). In this way, the system may inform the user of improper inputs and effectively guide the user to provide a proper input gesture.

As described above, an imaging system, such as the imaging system 100 of FIG. 1, may operate according to various states and/or execute various applications or other operations. The imaging system may also be configured to restrict operation responsive to predetermined conditions, such as an elapsed period of inactivity (e.g., in which no input is provided to the system and/or the system is not operated to perform any function other than background functions), a shut down and/or restart event, a selection to place the imaging system into a low power or secured mode, etc. During such restricted operation mode(s), the system may display a screensaver and/or lock screen via one or more displays. For example, a monitoring display (e.g., display 118 of FIG. 1, used to via an output from a scanning operation in some conditions) may display a screensaver (e.g., a generic screen) that prompts or otherwise instructs a user on how to unlock or “wake up” the system to perform further operations. The prompt/instructions may indicate that a swipe or other input to a touch input display (e.g., input device 115 of FIG. 1) will unlock or wake up the system.

FIG. 2A shows an example of a lock screen that may be shown on a touch-sensitive display 202, which may be an example of input device 115 of FIG. 1. As illustrated, a plurality of different “unlock” operations may be performed, each operation associated with a different direction of movement of a central user interface element 204 within an unlock element 206. For example, the central user interface element 204 may be the only interactive (e.g., selectable) user interface element on the display 202. In other examples, the central user interface element 204 may be the only interactive user interface element within the unlock element 206, and the display may provide other interactive user interface elements for performing operations that are unrelated to the unlocking of the system.

The unlock element 206 may include operation indicators in each of a plurality (e.g., four in the example illustrated in FIG. 2A) of directional locations around the central user interface element 204. Each indicator may include a graphical element identifying a different operation, mode, and/or application that is to be performed by the system responsive to moving the central user interface element 204 in the direction of that indicator and/or to within a threshold distance from a center of that indicator. For example, a first (e.g., emergency mode) indicator 208 may identify and engage an emergency mode operation of the imaging system. A second (e.g., user switch) indicator 210 may identify an operation to switch a user (e.g., change an active user of the system). A third (e.g., login and continue) indicator 212 may identify an operation to login (e.g., as a currently-active user) to the system and continue an examination or other scanning operation that is in progress. A fourth (e.g., shut down) indicator 214 may identify a shut down operation to power down/off the system.

FIG. 2B shows the display 202 responsive to a touch input from a user 216 directed to the central user interface element 204 to move the central user interface element toward the login and continue indicator 212. In some examples, the displayed user interface may react to inputs to the unlock element in real-time (e.g., prior to an operation associated with one of the indicators of the unlock element being selected). For example, a closest indicator(s) to the central user interface element during movement of the central user interface movement may be enhanced (e.g., enlarged, highlighted, recolored, distorted, etc.) to indicate that the user is moving toward selection of that indicator(s). Such enhancement may provide feedback regarding the user's input movements and/or create an easier-to-reach target for the user. In other examples, the user interface may remain unchanged, wherein only the central user interface element moves or changes responsive to the user input, and the remaining features of the unlock element 206 remain static.

The selection of an indicator (e.g., to effect an operation and/or launch an application associated with that indicator) may be made based on a position of the central user interface element as moved by the user 216 and/or a trajectory of a gesture that causes the movement of the central user interface element. In some examples, each indicator may have a selection region associated therewith. For example, selection region 218 (with a boundary shown by the dashed line) is shown as corresponding to the login and continue indicator 212. The login and continue indicator may be selected responsive to the gesture (e.g., and the resulting moved central user interface element) entering the selection region 218 (e.g., intersecting the selection region and/or selection region boundary), ending within the selection region 218 (e.g., the user lifting a finger or other input device while in the selection region), and/or staying within the selection region 218 for a threshold period of time (e.g., a dwell time). In examples where a dwell time is used to control the selection, the threshold period of time may either be static (e.g., the same for each indicator and/or based on a type of indicator associated with the selection window) or dynamically updated based on a state of the system (e.g., a number of indicators, an error state of the system, a user currently logged into the system, etc.) and/or the gesture input. For example, the threshold period of time may be different for different users, such that users may set preferences for a threshold dwell time for selection, or threshold dwell times may be set based on an experience level of the user (e.g., where more experienced users have lower threshold dwell times than less experienced users, with experience of a given user increasing with increasing frequency or number of inputs provided by the user to the system in some examples). The threshold dwell time may also dynamically change based on a trajectory of the gesture input. For example, the threshold dwell time may increase with an amount of time that the gesture is not within the selection region and/or with a level of complexity of a trajectory of the gesture input (e.g., a number of changes in direction of the gesture input, which may suggest that the user is not precisely controlling the user interface). As described herein, a single gesture input may be defined as starting when a user input is provided (e.g., “touches down” or otherwise engages the touch screen) in a region associated with the central user interface element. The gesture input may be defined as ending when the user input “lifts up” or otherwise no longer engages the touch screen. In some examples, engagement with the touch screen may include any engagement detected by the touch screen. In other examples, engagement with the touch screen may include engagement detected by the touch screen in the unlock element region (e.g., within a threshold distance of a user interface element—such as an indicator or the central user interface element—of the unlock element).

The selection region may also be static or dynamic based on one or more of the conditions described above with respect to the threshold dwell time. For example, the selection region may change in size or shape according to a user setting, a state of the imaging system, a number of other indicators, a direction/trajectory of gesture input, an experience of a user, etc.

Responsive to selecting an indicator, an imaging system operation and/or application associated with the selected indicator may be initiated or launched. In this way, a user may bypass a home screen, a login screen, or another user interface and engage the selected operation/application directly from the unlock element without providing any further inputs to the imaging system and without experiencing any delays associated with awaiting further inputs. Accordingly, from the unlock element, any one of the different operations/applications associated with the indicators displayed in the unlock element may be initiated or launched responsive to a single gesture input.

As will be described in more detail below, the number and type of indicators displayed within the unlock element may change based on a status or other operating condition of the imaging system, such that different indicators are displayed during different operating conditions of the imaging system. An example operating condition may include the imaging system being in an examination state, where an exam and/or other scanning operation is in progress. Another example operating condition may include a user and/or type of user logged into the system (or a determination that no user is currently logged into the system, such as after a shut down or restart of the system). Other example operating conditions may include currently-running applications, modes, types of examinations or scans being performed or last performed using the imaging system, whether or not the system has finished or is paused within an examination or other scanning operation, etc. In some examples, one or more indicators may always be shown regardless of the operating condition of the imaging system. For example, the emergency mode indicator 208 may be shown for any operating condition of the imaging system in order to provide quick access to emergency functions of the imaging system (e.g., an emergency shut down and/or reset of the system). In further examples, the emergency mode indicator 208 may be shown in the same position for any operating condition of the imaging system. In this way, a user may be more easily trained as to the location of the emergency mode indicator, increasing the ease-of-use of the associated emergency operations relative to other operations that are shifted according to system states. In an emergency mode, the imaging system may be operated with reduced functions relative to other modes (e.g., relative to a normal or default operating mode). In some examples, the emergency mode may not request authentication to utilize the imaging system. For example, the imaging system may be configured to start an exam, scan and acquire images, and store the images on an internal hard drive of the imaging system while operating in the emergency mode, even if no user has been authenticated. In the emergency mode, the imaging system may deny a user access to previous exam data and images of other patients for review purposes, in order to protect the data from un-authenticated users.

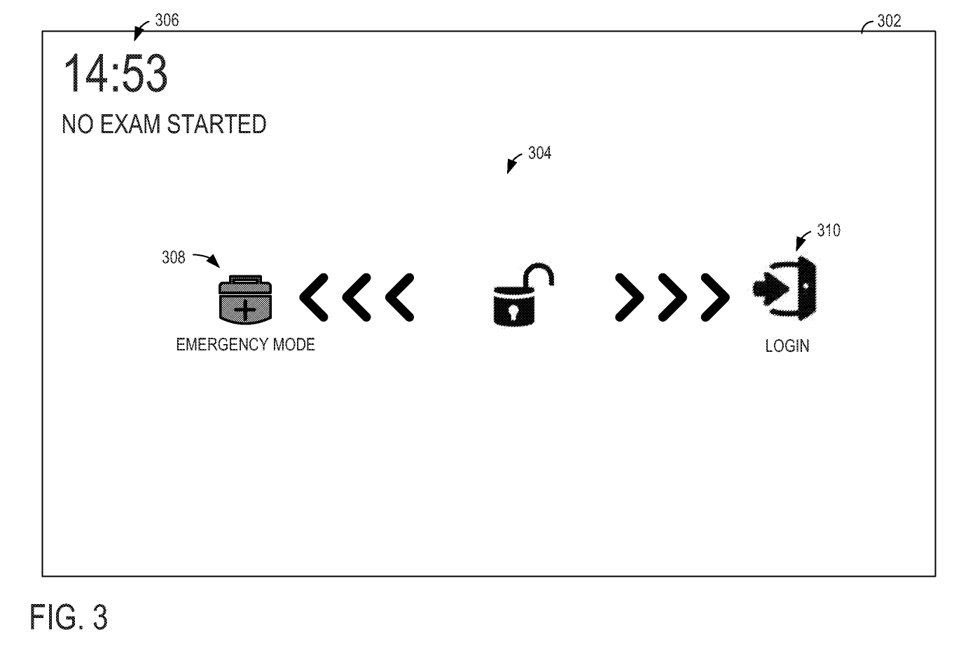

FIGS. 3-6 show examples of different unlock elements, which may be presented responsive to different operating states of the imaging system. For example, FIG. 3 shows a display 302 presenting an unlock element 304 that includes fewer (e.g., two) indicators than the unlock element 206 of FIG. 2. As shown by the status indicator 306, the unlock element 304 may be presented responsive to the system being in a “no examination” state in which no examination or other scanning operation is being performed. The unlock element 304 of FIG. 3 may also correspond to a system state in which no user is currently logged in to the system. Accordingly, the only indicators displayed in the unlock element 304 may include an emergency mode indicator 308 and a login indicator 310.

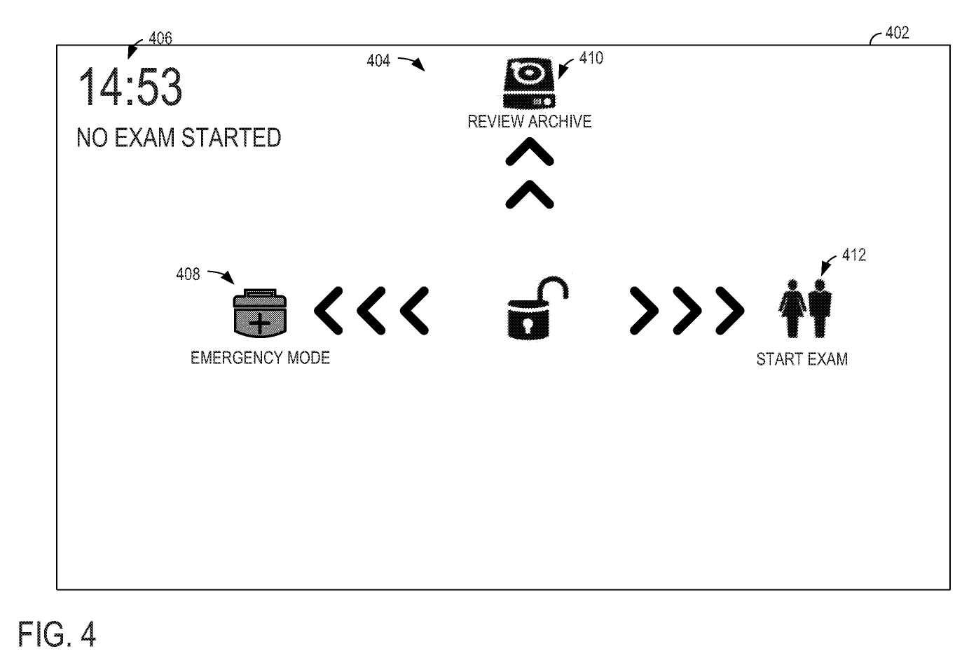

FIG. 4 shows a display 402 presenting an unlock element 404 that includes three indicators for unlock operations. As shown by the status indicator 406, the unlock element 404 may be presented responsive to the system being in a “no examination” state, similarly to unlock element 304 of FIG. 3. However, in the example of FIG. 4, a user may be logged in to the system, resulting in the display of an emergency indicator 408, an archive review indicator 410, and an exam start indicator 412. The archive review indicator 410, when selected, may launch an application that provides access to an archive of all and/or a subset of examinations performed by the system. The archive may include images acquired via the past examinations and/or annotations or other data collected or input during the past examinations. The exam start indicator 412, when selected, may initiate an examination and/or launch an examination application that provides access to an examination setup or other user interface for performing an examination or other scanning operation with the imaging system.

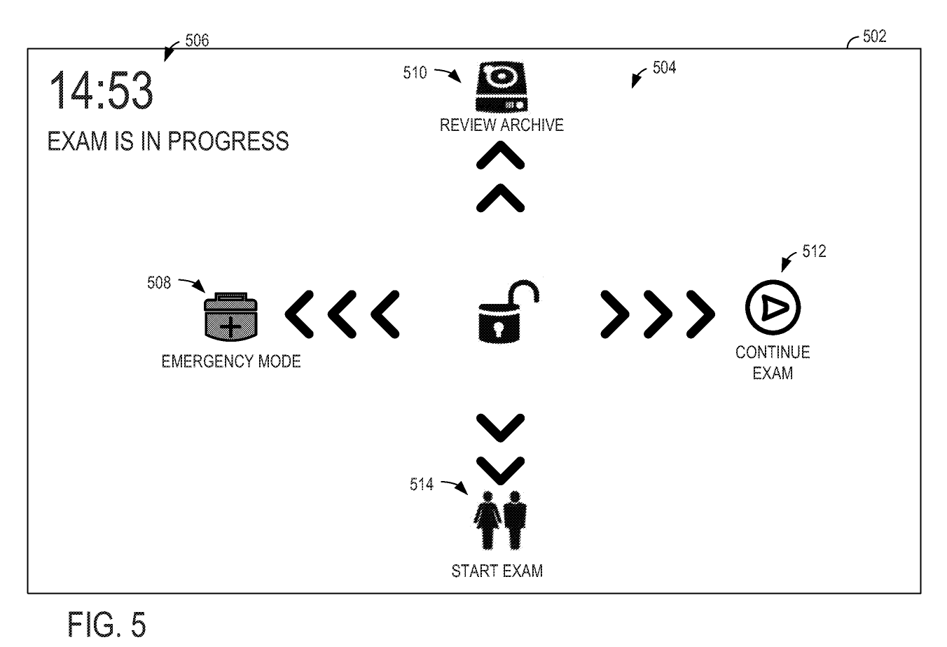

FIG. 5 shows a display 502 presenting an unlock element 504 that includes four indicators for unlock operations. As shown by the status indicator 506, the unlock element 504 may be presented responsive to the system being in an examination state (e.g., while an examination or other scanning operation is in progress), similarly to unlock element 206 of FIGS. 2A and 2B. The indicators of unlock element 504 may be different from those of unlock element 206 of FIGS. 2A and 2B due to a user being logged into the system and/or due to the type of user logged into the system. For example, different users may have different levels of authority or permissions (e.g., access to different applications or operations of the imaging system) and/or different preferences, which may control the indicators presented within an unlock user interface element while that user is logged into the imaging system. The unlock element 504 includes an emergency mode indicator 508, an archive review indicator 510, an examination continue indicator 512, and an exam start indicator 514. The emergency mode indicator, archive review indicator, and exam start indicator may be the same or similar to the correspondingly-named indicators of unlock element 404 of FIG. 4. Accordingly, the description of these elements provided with respect to FIG. 4 may also apply to these elements as they appear in FIG. 5. The examination continue indicator 512 may be selected to resume the examination that is in progress (e.g., the examination indicated to be in progress via the status indicator 506). In the example of FIG. 5, the system may be able to continue the exam without requesting log in credentials from the user. For example, the user may have logged into the system within a period of time that is less than an automated logout threshold and/or the currently logged in user may have user preferences and/or privileges that keep the user's credentials active even after the system enters a locked state, enters a sleep state, and/or is restarted.

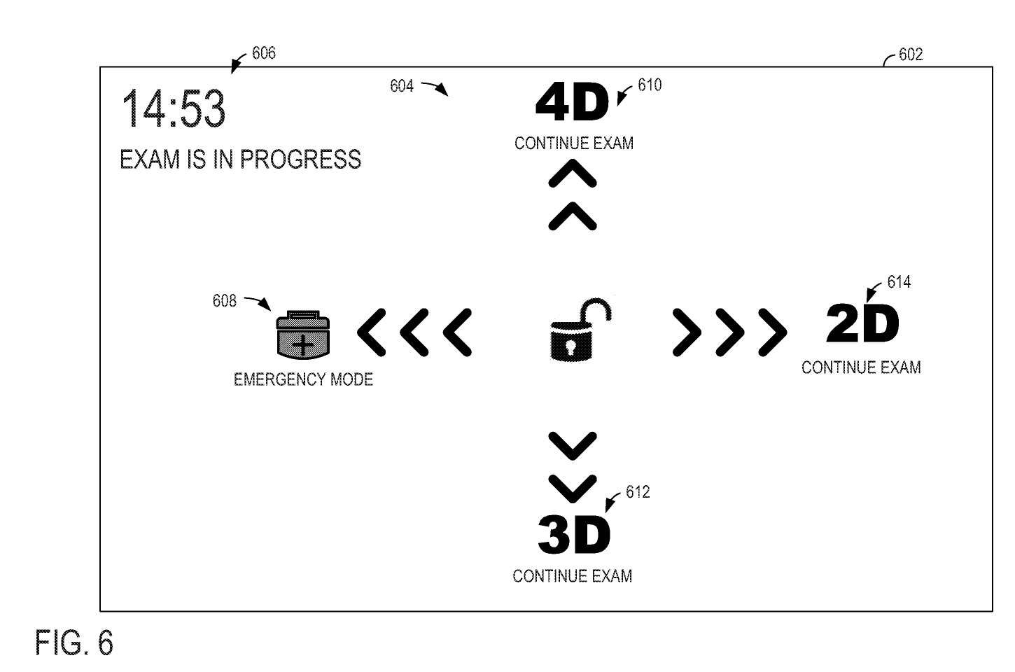

FIG. 6 shows a display 602 presenting an unlock element 604 including indicators that provide multiple options for continuing an examination or other scanning operation that is already in progress (e.g., as indicated by status indicator 606). As with the prior examples of unlock elements, unlock element 604 includes an emergency mode indicator 608, which may correspond to the emergency mode indicators described above with respect to FIGS. 2A-5. The unlock element 604 also includes a 4D examination continue indicator 610, a 3D examination continue indicator 612, and a 2D examination continue indicator 614. As described above with respect to FIG. 1, an examination provided by an imaging system, such as system 100 of FIG. 1, may include capturing image data (e.g., of an anatomical feature of a patient) and combining and restructuring the image data into a composite image (e.g., of the anatomical feature). The composite image may include one or more two-dimensional (2D) images, a three-dimensional (3D) volume, or a 3D volume over time (four-dimensional, 4D). Accordingly, the selection of the 4D indicator 610 may control the system to continue the examination by generating (e.g., using image data corresponding to acquired ultrasound information) one or more composite images including a 3D volume over time. The selection of the 3D indicator 612 may control the system to continue the examination by generating (e.g., using image data corresponding to acquired ultrasound information) one or more composite images including a 3D volume. The selection of the 2D indicator 614 may control the system to continue the examination by generating (e.g., using image data corresponding to acquired ultrasound information) one or more composite images including one or more 2D images.

In each of the above examples, indicators for different unlock functions are positioned in different cardinal directions relative to a central user interface element. For example, when two indicators are included in the unlock element, the two indicators are positioned on opposite sides of the central user interface element, along an axis that passes through the central user interface element (e.g., at 90° and 270° positions relative to the central user interface element, where a top of the display corresponds to a 0° position). In the illustrated examples, when three indicators are included in the unlock element, two of the indicators are positioned on opposite sides of the central user interface element along a first axis that passes through the central user interface element (e.g., as in the two indicator example, at 90° and 270° positions relative to the central user interface element) and a third indicator is positioned along a second axis that passes through the central user interface element and is perpendicular to the first axis (e.g., at the 0° position). In some examples, the indicators are positioned an equal distance from an adjacent indicator and each of the indicators are positioned at the same distance from the central user interface element. In other examples, such as when the unlock element occupies a rectangular or ovular space (e.g., where a height of the unlock element is more than a threshold amount larger or smaller than a width of the unlock element), indicators along a same axis may be spaced from the central user interface element by the same amount of distance, but indicators along different axes may be spaced from the central user interface element by a different amount of distance. For example, if an aspect ratio of the display results in the presentation of an unlock element that has a shorter height than width, indicators positioned on opposing sides of the central user interface element (e.g., at 90° and 270° positions) may be further away from the central user interface element than indicators positioned on top of or below the user interface element (e.g., at 0° and 180° positions). The distance between a given indictor and the central user interface element may thereby be a function of an aspect ratio of the display and/or an aspect ratio of the unlock element (e.g., where the size of the unlock element is not directly tied to the aspect ratio of the display, such as when other elements are displayed on the display and restrict the positioning and/or size of the unlock element).

The positioning of the indicators around the central user interface element may be selected based on a number of indicators being presented. For example, positions for each indicator may be selected in order to evenly space the indicators from one another around a periphery of the central user interface element in order to facilitate the differentiation of gestures to select each indicator. In the illustrated examples, the even spacing of the indicators may be associated with a predefined grid or other layout structure. For example, a predefined layout may define positions for a threshold number of indicators, and indicators may fill the positions in a predefine order (e.g., a first indicator is displayed in a first position, a second indicator is displayed in a second position, etc.). The system may be configured to limit the display of indicators based on the number of predefined positions in the layout. For example, each indicator that may be displayed for a given user and/or operating state of the imaging system may be associated with a level of relevancy or importance for the given user and/or operating state, and the indicators may populate the predefined positions in an order based on the relative level of relevancy or importance (e.g., such that the positions are populated with the most relevant/important indicators first). In some examples, the relevancy and/or importance may be user-selected (e.g., as a user preference).

The above scenario describes an automated placement of indicators in the unlock element. In some examples, the placement of indicators may be based on user preferences input by a user. For example, a user may select indicators to be presented in the unlock element and/or positions for the selected indicators within a user preferences application. Subsequently, when an unlock element is presented for that user (e.g., while that user is logged in and/or when that user was the last logged in user), the unlock element may include the indicators selected by the user in positioned according to the user's preferences. Where the user does not select a position for a given indicator(s), the indicator(s) may be automatically positioned as described above. A user may select a global indicator preference (e.g., where the same indicators/positions are maintained regardless of the state of the imaging system) and/or state-based indicator preferences (e.g., where indicators/positions are selected on a per-state basis for each of one or more possible states of the imaging system, such as an exam in progress state, no examination state, user logged in state, user logged out state, etc.). In some examples, a user (e.g., a user with authorization/privileges above a threshold) may define a default indicator selection/positioning for the imaging system, which may be utilized for the system whenever a user has not defined his/her own preferences and/or when no user is logged into the system.

As discussed above with respect to FIG. 1, an imaging system may have multiple displays, such as a display device 118 for displaying images and/or other results of an examination or other scanning operation, and a touch-sensitive display of an input device 115. Accordingly, in some examples, different images or interfaces may be displayed via different displays. While FIGS. 2A-6 illustrated example lock screens for one display of an imaging system (e.g., displayed via a touch-sensitive display), FIGS. 7A and 7B show example images that may be shown via a second display of the imaging system. For example, if the displays of FIGS. 2A-6 correspond to input device 115 of FIG. 1, display 700 of FIGS. 7A and 7B may correspond to display 118 of FIG. 1. In other examples, the displays of FIGS. 2A-6 may correspond to display 118 of FIG. 1, while the display 700 of FIGS. 7A and 7B correspond to a display of input device 115 of FIG. 1. It is to be understood that an imaging system may include any suitable number of displays, including one or more of the displays of FIGS. 2A-6 and display 700 of FIGS. 7A and 7B.

FIG. 7A shows a first example image 702a that may be presented via display 700 while an associated imaging system is in a locked or sleep state. For example, the image 702a may be displayed while an unlock element is displayed via another display device. The image 702a may include a prompt 704a to instruct a user on how to unlock the associated imaging system. For example, the prompt may indicate that the user is to provide an input (e.g., a “swipe”) to another touch screen to being operation of the imaging system.

FIG. 7A shows a second example image 702b that may be presented via display 700 while an associated imaging system is in a locked or sleep state. For example, the image 702b may be displayed while an unlock element is displayed via another display device. Similarly to image 702a, image 702b may include a prompt 704b to instruct a user on how to unlock the associated imaging system. Image 702b may also include a status indicator 706 showing that the imaging system is currently performing an examination. The status indicator 706 may include an icon that is similar to or the same as an icon associated with a scan in the unlock element (e.g., as shown in FIG. 4) in order to provide a bridge between the interfaces of the two displays. By viewing the similar icons, a user of the system may quickly come to associate the symbol within the status indicator 706 with the performance of an examination.

In order to provide further ties between the display 700 and an associated touch screen showing an unlock user interface element, an image displayed on display 700 may have a background color and/or pattern/image that is the same as the associated touch screen. In this way, the two displays may present an image with the same background color, pattern, and/or image at the same time. This color, pattern, and/or image may also indicate a state of the system. For example, each of the unlock screens shown in FIGS. 2A-6 may be displayed with a different background color, pattern, and/or image, and the image displayed via an associated display (e.g., display 700) may be dynamically updated to match the current unlock element's background feature. After the imaging system has been unlocked (e.g., via input to an unlock element of an associated touchscreen), the display 700 may revert to displaying a selected application of the imaging system (e.g., a scanning application, a login screen, etc.).

FIGS. 8-10 show example methods of operating a touch screen of an imaging system, such as input device 115 of imaging system 100 of FIG. 1, under different operating conditions. FIG. 8 is a flow chart of an example method 800 of operating the imaging system and the touch screen in a first condition, where the imaging system has user authentication enabled (e.g., where a user must log in to the system in order to start or continue using the system). At 802, the method includes booting up the imaging system. The imaging system may be booted from a cold start (e.g., where the system was fully shut down) or from a hibernate or sleep state or other low power mode (e.g., where only some resources of the system were shut down). At 804, the method includes displaying an unlock element via a touchscreen. As indicated at 806, the unlock element may include a plurality of indicators representing different operations and/or applications that may be launched via input to the unlock element. The unlock element displayed at 804 may correspond to unlock element 206 of FIGS. 2A and 2B in the example method.

At 808, the method includes monitoring the touchscreen for input. For example, the method may include monitoring the touchscreen for gesture input, which may include determining a direction of input directed to a central user interface element of the unlock element, as described above with respect to FIG. 2B. Responsive to detecting input to the touchscreen, the method includes determining if input selecting an emergency mode is detected, as indicated at 810. If input selecting an emergency mode is detected (e.g., “YES” at 810), the method proceeds to 812 to enter an emergency mode. Entering the emergency mode may include performing an emergency scan, as indicated at 814, performing an emergency shut down, as indicated at 816, and/or performing other emergency operations. After exiting an emergency mode and/or performing an emergency operation, the method may return to a locked state and display the unlock element as indicated at 804. In some examples, where the emergency operation includes an emergency shut down, the method may instead return to 802 to boot up the imaging system (e.g., after a boot operation is triggered following the emergency shut down).

If input selecting the emergency mode is not detected (e.g., “NO” at 810), the method proceeds to 818 to determine whether input selecting a shut down indicator is detected. If input selecting the shut down indicator is detected (e.g., “YES” at 818), the method proceeds to 820 to turn off the imaging system (e.g., to shut down the imaging system) and then returns (e.g., to 802 to boot up the imaging system responsive to a trigger received after the shut down). If input selecting the shut down indicator is not detected (e.g., “NO” at 818), the method proceeds to 822 to determine if input selecting a user authentication operation is detected. If input selecting a user authentication operation is not detected (e.g., “NO” at 822), the method optionally proceeds to 824 (e.g., if input is detected that does not select one of the available options) to display an error message indicating that an improper input was detected. The method then or otherwise returns to continue displaying the unlock element at 804 and monitor for input.

If input selecting a user authentication operation is detected (e.g., “YES” at 822), the method proceeds to 826 to perform user authentication. For example, a login screen or other mechanism (e.g., bioscanner) may be engaged in order to receive authentication input from the user. Responsive to authenticating the user and/or confirming that the user is logged in to the system, the method includes entering (and operating according to) a nominal scan mode, as indicated at 828. At 830, the method includes determining whether inactivity is detected (e.g., inactivity that is greater than a threshold associated with a display of an unlock screen). If inactivity is not detected (e.g., a period of inactivity that is greater than the threshold is not detected, “NO” at 830), the method returns to 828 to continue operating the imaging system according to a nominal scan mode. If inactivity is detected (e.g., a period of inactivity that is greater than the threshold is detected, “YES” at 830), the method proceeds to 832 to display an unlock element via the touchscreen. Since a scanning operation is in progress (e.g., initiated at 828), the unlock element displayed at 832 may correspond to the unlock element 304 of FIG. 3, and may include emergency mode, shut down, current user authentication and new user authentication indicators, as indicate at 834. Upon displaying the unlock element, the method may return to 808 to monitor the touchscreen for input to the updated unlock element.

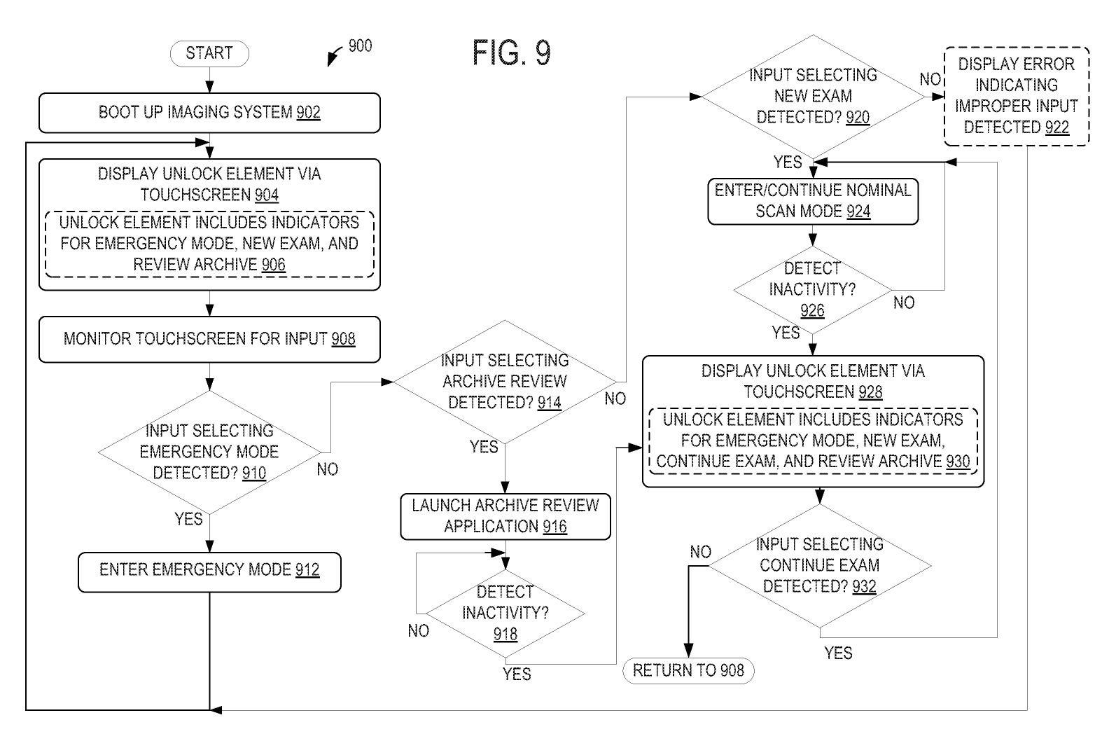

FIG. 9 shows an example method 900 of operating an imaging system and associated touchscreen in a second condition where the imaging system does not have an authentication mode enabled. At 902, the method includes booting up the imaging system. The boot up operation may be similar to that performed at 802 of FIG. 8 in some examples. At 904, the method includes displaying an unlock element via a touch screen, where the unlock element includes indicators for an emergency mode, a new exam, and a review archive, as indicated at 906. For example, the unlock element illustrated in FIG. 4 (or a similar unlock element with similar user interface elements) may be displayed at 904 in some examples. At 908, the method includes monitoring the touchscreen for input (e.g., similarly to the monitoring performed at 808 of FIG. 8). At 910, the method includes determining if input selecting the emergency mode indicator is detected. If input selecting an emergency mode is detected (e.g., “YES” at 910), the method proceeds to 912 to enter an emergency mode. Entering the emergency mode may include performing an emergency scan, performing an emergency shut down, and/or performing other emergency operations. After exiting an emergency mode and/or performing an emergency operation, the method may return to a locked state and display the unlock element as indicated at 904. In some examples, where the emergency operation includes an emergency shut down, the method may instead return to 902 to boot up the imaging system (e.g., after a boot operation is triggered following the emergency shut down).

If input selecting the emergency mode is not detected (e.g., “NO” at 910), the method proceeds to 914 to determine whether input selecting an archive review indicator is detected. If input selecting the archive review indicator is detected (e.g., “YES” at 914), the method proceeds to 916 to launch an archive review application. In the archive review application, as described earlier in the disclosure, data from prior scans may be made available for review (e.g., displayed or otherwise presented). At 918, the method includes determining if a period of inactivity (e.g., exceeding a threshold) is detected. If not, the method returns to continue monitoring for inactivity (e.g., to continue executing the archive review application. It is to be understood that some loops in the methods described herein may be exited by user input selecting an alternative application. For example, a user may launch another application or otherwise change operation of the imaging system while the system is unlocked. If a period of inactivity (e.g., exceeding a threshold) is detected (e.g., “YES” at 918), the method proceeds to 928 to display an updated unlock element, which will be described in more detail below.

If input selecting the archive review is not detected (e.g., “NO” at 914), the method proceeds to 920 to determine whether input selecting a new exam is detected. If not (e.g., “NO” at 920), the method optionally proceeds to 922 to display an error (as described above with respect to FIG. 8), then returns to continue monitoring for input. If input selecting a new exam is detected (e.g., “YES” at 920), the method proceeds to 924 to enter a nominal scan mode (e.g., as described above with respect to FIG. 8). Similarly to method 800, method 900 includes detecting a period of inactivity (e.g., that exceeds a threshold), and displaying an unlock element responsive to detecting the inactivity at 928. However, due to the conditions of the imaging system in the example of FIG. 9, the unlock element displayed at 928 includes indicators for an emergency mode, a new exam, a continued exam, and an archive review, as indicated at 930. For example, the unlock element 504 of FIG. 5 may be an example of an unlock element displayed at 930.

The method includes determining if input selecting the continue exam indicator is detected at 932. If so (e.g., “YES” at 932), the method returns to 924 to continue the nominal scan mode. If input selecting the continue exam indicator is not detected at 932, the method returns to 908 to continue monitoring for other inputs.

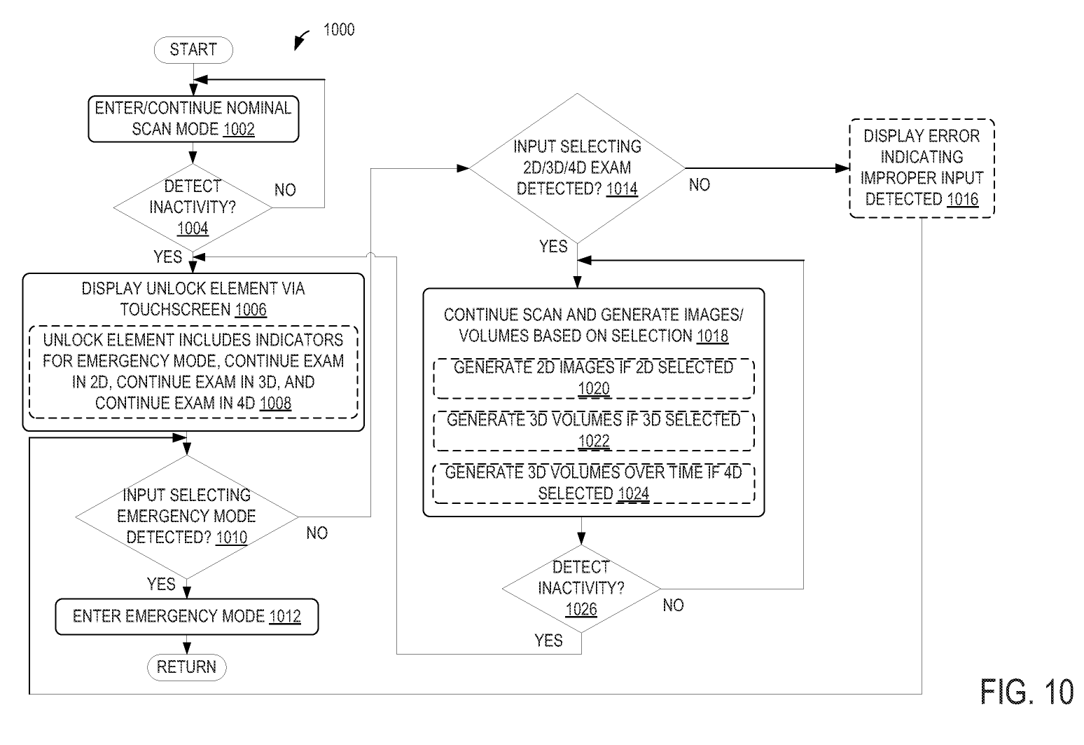

FIG. 10 shows a flow chart for a method 1000 of operating an imaging system and associated touchscreen under operating conditions in which an authentication mode is not enabled and the system is configured to invoke different scanning modes quickly. Method 1000 includes entering and/or continuing a nominal scanning mode at 1002 (it is to be understood that this mode may be entered responsive to booting up the imaging system and providing input to a displayed unlock element). At 1004, the method includes determining if inactivity (e.g., above a threshold) is detected. If not (e.g., “NO” at 1004), the method returns to 1002 to continue the nominal scanning mode. If inactivity (e.g., above a threshold) is detected (e.g., “YES” at 1004), the method proceeds to 1006 to display an unlock element via the touchscreen. The unlock element may include indicators for an emergency mode, a continue exam in 2D mode, a continue exam in 3D mode, and a continue exam in 4D mode, as indicated at 1008.

At 1010, the method includes determining if input selecting the emergency mode is detected. If so (e.g., “YES” at 1010), the method includes proceeding to enter the emergency mode, as indicated at 1012. If input selecting the emergency mode is not detected (e.g., “NO” at 1010), the method proceeds to 1014 to determine if input selecting one of the 2D/3D/4D indicators is detected. If not (e.g., “NO” at 1014), the method optionally proceeds to 1016 to display an error indicating that improper input was detected, then returns to continue monitoring for input. If input selecting one of the 2D/3D/4D indicators is detected (e.g., “YES” at 1014), the method proceeds to 1018 to continue the scanning operation and generate images and/or volumes based on the selection of the mode. For example, as indicated at 1020, the method includes generating 2D images if the 2D indicator is selected. As indicated at 1022, the method includes generating 3D volumes if the 3D indicator is selected. As indicated at 1024, the method includes generating representations of 3D volumes over time if the 4D indicator is selected.

At 1026, the method includes determining if inactivity (e.g., above a threshold) is detected. If so (e.g., “YES” at 1026), the method returns to 1006 to display the unlock element including the emergency mode indicator and scan mode indicators. If inactivity (e.g., above a threshold) is not detected (e.g., “NO” at 1026), the method returns to 1018 to continue the scanning and generation of images according to the input selection detected at 1014.

FIG. 11 schematically shows example input and output devices for an imaging system 1100. A first display 1102 may be an example of display device 118 of FIG. 1 and/or display 700 of FIGS. 7A and 7B. The first display 1102 may be configured to output images, volumes, and/or other data regarding an examination or other scanning operation. The first display 1102 may also present a screensaver, such as those described above with respect to FIGS. 7A and 7B. The imaging system 1100 may include a second display 1104, which may include a touchscreen for receiving input. The second display 1104 may be an example of input device 115 of FIG. 1 and/or displays 202, 302, 402, 502, and 602 of FIGS. 2A-6. The second display 1104 may display an unlock element (e.g., while the first display presents an associated screensaver that prompts the user to unlock the system via the second display). The imaging system 1100 may also include other input mechanisms, such as trackball 1106, buttons 1108, knobs 1110, and keyboard 1112. Input provided to these additional input mechanisms may be provided to control the imaging system and/or to control the output of either the first display or the second display. In some examples, during a locked or sleep state of the imaging system, the system may only detect and/or respond to inputs received via the touchscreen of the second display 1104. By displaying an unlock element via the second display, as described herein, operations that may be performed via actuation of the buttons, knobs, or other input mechanisms may be executed via touch input to the unlock element presented by the second display.

In this way, many operations of an imaging system may be efficiently accessed via a single input made to a user interface element that is displayed while the imaging system is in a locked, sleep, low power, or other reduced operation state. Different unlock elements may be provided based on a state of the imaging system, user preferences, and/or other conditions. In this way, the operations made available via the unlock element may be most relevant to a current user/condition of the imaging system. A technical effect of presenting an unlock element and launching operations of an imaging system therefrom is decreasing startup delays for the imaging system by enabling a user to bypass a home screen or other interface that may be otherwise presented after performing an unlock operation in other systems. Furthermore, the use of a touchscreen to unlock the system may help to familiarize the user with other touchscreen inputs that may be made during operation of the imaging system.

The systems and methods described above also provide for an imaging system including a touch-sensitive display device, a controller, and a storage device storing instructions executable by the controller to: display, via the touch-sensitive display device, a user interface comprising a central user interface element and a plurality of operation indicators positioned around a periphery of the central user interface element, and, responsive to user input moving the central user interface element in a direction toward a first indicator of the operation indicators, executing an application or operation associated with the first indicator upon intersecting a selection region for the first indicator. In a first example of the imaging system, the plurality of operation indicators may additionally or alternatively include one or more of a perform new imaging scan indicator, a continue imaging scan indicator, a review scan archive indicator, and a scanning mode selection indicator. A second example of the imaging system optionally includes the first example, and further includes the imaging system, wherein the scanning mode selection indicator includes one or more of: a two-dimensional scanning mode indicator selectable to perform a scan that generates two-dimensional images, a three-dimensional scanning mode indicator selectable to perform a scan that generates three-dimensional volumes, and a four-dimensional scanning mode indicator selectable to perform a scan that generates representations of three-dimensional volumes over time. A third example of the imaging system optionally includes one or both of the first and the second examples, and further includes the imaging system, wherein the plurality of operation indicators further include one or more of an emergency mode indicator and a user authentication indicator. A fourth example of the imaging system optionally includes one or more of the first through the third examples, and further includes the imaging system, wherein the instructions are further executable to select the number and type of operation indicators included in the user interface based on user preferences input by a user that is logged into the imaging system. A fifth example of the imaging system optionally includes one or more of the first through the fourth examples, and further includes the imaging system, wherein the instructions are further executable to select the number and type of operation indicators included in the user interface based on a current state of the imaging system. A sixth example of the imaging system optionally includes one or more of the first through the fifth examples, and further includes the imaging system, wherein the instructions are further executable to position the operation indicators around the central user interface element based on a predefined layout and a number of operation indicators included in the user interface. A seventh example of the imaging system optionally includes one or more of the first through the sixth examples, and further includes the imaging system, wherein, for a user interface that includes at least two operation indicators, a first operation indicator is positioned on an opposite side of the central user interface element from a second operation indicator, the first and second operation indicators being positioned along a first axis that passes through the central user interface element. An eighth example of the imaging system optionally includes one or more of the first through the seventh examples, and further includes the imaging system, wherein, for a user interface that includes at least three operation indicators, a third operation indicator is positioned along a second axis that passes through the central user interface element and is perpendicular to the first axis, the third operation indicator being spaced from the first operation indicator and the second operation indicator by the same amount of distance. A ninth example of the imaging system optionally includes one or more of the first through the eighth examples, and further includes the imaging system, wherein the touch-sensitive display device is a first display device, the imaging system further comprising a second display device configured to display a different user interface than the user interface displayed via the first display device.

The methods and systems described above also provide for a method including displaying, via a touch-sensitive display device of an imaging system, a user interface comprising a central user interface element and a plurality of operation indicators positioned around a periphery of the central user interface element, detecting user input at the touch-sensitive display device, and responsive to the user input moving the central user interface element in a direction toward a first indicator of the operation indicators, executing an application or operation associated with the first indicator upon intersecting a selection region for the first indicator. In a first example of the method, the plurality of operation indicators may additionally or alternatively include one or more of a perform new imaging scan indicator, a continue imaging scan indicator, a review scan archive indicator, and a scanning mode selection indicator. A second example of the method optionally includes the first example, and further includes the method, wherein the scanning mode selection indicator includes one or more of: a two-dimensional scanning mode indicator selectable to perform a scan that generates two-dimensional images, a three-dimensional scanning mode indicator selectable to perform a scan that generates three-dimensional volumes, and a four-dimensional scanning mode indicator selectable to perform a scan that generates representations of three-dimensional volumes over time. A third example of the method optionally includes one or both of the first and the second examples, and further includes the method, wherein the plurality of operation indicators further include one or more of an emergency mode indicator and a user authentication indicator. A fourth example of the method optionally includes one or more of the first through the third examples, and further includes the method, wherein the number and type of operation indicators included in the user interface is selected based on whether or not a user authentication mode is active on the imaging system. A fifth example of the method optionally includes one or more of the first through the fourth examples, and further includes the method, wherein the number and type of operation indicators included in the user interface is selected based on whether or not an examination is in progress by the imaging system. A sixth example of the method optionally includes one or more of the first through the fifth examples, and further includes the method, wherein the number and type of operation indicators included in the user interface is selected based on one or more of permissions and preferences set for a user that is currently logged into the imaging system. A seventh example of the method optionally includes one or more of the first through the sixth examples, and further includes the method, further including changing a size of the selection region based on one or more of a state of the imaging system and user preferences for a user that is currently logged into the imaging system. A seventh example of the method optionally includes one or more of the first through the sixth examples, and further includes the method, wherein the operation indicators are positioned around the central user interface element based on a predefined layout and a number of operation indicators included in the user interface.

The systems and methods described above also provide for an imaging system including a first display device, a second display device, the second display device including a touch-sensitive input mechanism for receiving touch-based gesture input, a controller, and a storage device storing instructions executable by the controller to: display, via the first display device, an image prompting a user to unlock the imaging system while the imaging system is in a locked state, display, via the second display device, a user interface comprising a central user interface element and a plurality of operation indicators positioned around a periphery of the central user interface element, the user interface being displayed via the second display device while the image prompting the user to unlock the imaging system is displayed via the first display device, and the plurality of operation indicators including an emergency mode operation indicator for entering an emergency mode of operation at the imaging system, and, responsive to user input moving the central user interface element in a direction toward a first indicator of the operation indicators, executing an application or operation associated with the first indicator upon intersecting a selection region for the first indicator.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An imaging system comprising:
a touch-sensitive display device;
a controller; and
a storage device storing instructions executable by the controller to:
display, via the touch-sensitive display device, a user interface comprising a central user interface element and a plurality of operation indicators positioned around a periphery of the central user interface element; and
responsive to user input moving the central user interface element in a direction toward a first indicator of the operation indicators, executing an application or operation associated with the first indicator upon intersecting a selection region for the first indicator.

2. The imaging system of claim 1, wherein the plurality of operation indicators include one or more of a perform new imaging scan indicator, a continue imaging scan indicator, a review scan archive indicator, and a scanning mode selection indicator.

3. The imaging system of claim 2, wherein the scanning mode selection indicator includes one or more of:
a two-dimensional scanning mode indicator selectable to perform a scan that generates two-dimensional images,
a three-dimensional scanning mode indicator selectable to perform a scan that generates three-dimensional volumes, and
a four-dimensional scanning mode indicator selectable to perform a scan that generates representations of three-dimensional volumes over time.

4. The imaging system of claim 2, wherein the plurality of operation indicators further include one or more of an emergency mode indicator and a user authentication indicator.

5. The imaging system of claim 1, wherein the instructions are further executable to select the number and type of operation indicators included in the user interface based on user preferences input by a user that is logged into the imaging system.

6. The imaging system of claim 1, wherein the instructions are further executable to select the number and type of operation indicators included in the user interface based on a current state of the imaging system.

7. The imaging system of claim 1, wherein the instructions are further executable to position the operation indicators around the central user interface element based on a predefined layout and a number of operation indicators included in the user interface.

8. The imaging system of claim 7, wherein, for a user interface that includes at least two operation indicators, a first operation indicator is positioned on an opposite side of the central user interface element from a second operation indicator, the first and second operation indicators being positioned along a first axis that passes through the central user interface element.

9. The imaging system of claim 8, wherein, for a user interface that includes at least three operation indicators, a third operation indicator is positioned along a second axis that passes through the central user interface element and is perpendicular to the first axis, the third operation indicator being spaced from the first operation indicator and the second operation indicator by the same amount of distance.

10. The imaging system of claim 1, wherein the touch-sensitive display device is a first display device, the imaging system further comprising a second display device configured to display a different user interface than the user interface displayed via the first display device.

11. A method comprising:
displaying, via a touch-sensitive display device of an imaging system, a user interface comprising a central user interface element and a plurality of operation indicators positioned around a periphery of the central user interface element;
detecting user input at the touch-sensitive display device; and
responsive to the user input moving the central user interface element in a direction toward a first indicator of the operation indicators, executing an application or operation associated with the first indicator upon intersecting a selection region for the first indicator.

12. The method of claim 11, wherein the plurality of operation indicators include one or more of a perform new imaging scan indicator, a continue imaging scan indicator, a review scan archive indicator, and a scanning mode selection indicator.

13. The method of claim 12, wherein the scanning mode selection indicator includes one or more of:
a two-dimensional scanning mode indicator selectable to perform a scan that generates two-dimensional images,
a three-dimensional scanning mode indicator selectable to perform a scan that generates three-dimensional volumes, and
a four-dimensional scanning mode indicator selectable to perform a scan that generates representations of three-dimensional volumes over time.

14. The method of claim 12, wherein the plurality of operation indicators further include one or more of an emergency mode indicator and a user authentication indicator.

15. The method of claim 11, wherein the number and type of operation indicators included in the user interface is selected based on whether or not a user authentication mode is active on the imaging system.

16. The method of claim 11, wherein the number and type of operation indicators included in the user interface is selected based on whether or not an examination is in progress by the imaging system.

17. The method of claim 11, wherein the number and type of operation indicators included in the user interface is selected based on one or more of permissions and preferences set for a user that is currently logged into the imaging system.

18. The method of claim 11, further comprising changing a size of the selection region based on one or more of a state of the imaging system and user preferences for a user that is currently logged into the imaging system.

19. The method of claim 11, wherein the operation indicators are positioned around the central user interface element based on a predefined layout and a number of operation indicators included in the user interface.

20. An imaging system comprising:
a first display device;
a second display device, the second display device including a touch-sensitive input mechanism for receiving touch-based gesture input;
a controller; and
a storage device storing instructions executable by the controller to:
display, via the first display device, an image prompting a user to unlock the imaging system while the imaging system is in a locked state;
display, via the second display device, a user interface comprising a central user interface element and a plurality of operation indicators positioned around a periphery of the central user interface element, the user interface being displayed via the second display device while the image prompting the user to unlock the imaging system is displayed via the first display device, and the plurality of operation indicators including an emergency mode operation indicator for entering an emergency mode of operation at the imaging system; and
responsive to user input moving the central user interface element in a direction toward a first indicator of the operation indicators, executing an application or operation associated with the first indicator upon intersecting a selection region for the first indicator.
